(19) United States
(12) Patent Application Publication
(10) Pub. No.: US 2022/0126067 A1
(43) Pub. Date: Apr. 28, 2022

(54) EXTENSION CATHETER AND METHOD FOR PRODUCING SAME

(71) Applicant: KANEKA CORPORATION, Osaka-shi, Osaka (JP)

(72) Inventor: Toshiya KISA, Settsu-shi (JP)

(73) Assignee: KANEKA CORPORATION, Osaka-shi, Osaka (JP)

(21) Appl. No.: 17/423,420

(22) PCT Filed: Jan. 29, 2020
(86) PCT No.: PCT/JP2020/003216

§ 371 Date: Jul. 15, 2021

(30) Foreign Application Priority Data
Feb. 6, 2019 (JP) 2019-019998

(51) Int. Cl. A61M 25/09; A61M 25/00

(52) U.S. Cl. CPC A61M 25/0905; A61M 2025/0024; A61M 2205/0216; A61M 25/0023

(57) ABSTRACT

The purpose of the present invention is to provide an extension catheter having excellent breaking strength and a method of producing the same. The extension catheter includes a tubular portion, a tapered portion disposed proximally relative to the tubular portion, and a linear member fixed to the tubular portion and the tapered portion, the extension catheter satisfying Formula (1): 1.1T₂≤T₁≤6T₂, wherein T₁ represents a thickness (mm) of the tubular portion at a first position located 1 mm to a distal end of the tapered portion, the position being a position where the linear member is present, and T₂ represents a thickness (mm) of the tubular portion at a second position opposite to the first position.

### TECHNICAL FIELD

The present invention relates to an extension catheter and a method of producing the same.

### BACKGROUND ART

For ischemic heart diseases like angina pectoris and cardiac infarction, there has been conducted percutaneous coronary intervention (PCI) including expanding a stenosed part in a coronary artery of a heart with use of an intravascular treatment instrument such as a stent or a balloon to increase a blood flow. Typically in PCI, a tip end of a tubular guiding catheter is inserted to an ostium of the coronary artery and is indwelt, and the intravascular treatment instrument is then delivered through the guiding catheter, for easier insertion of the intravascular treatment instrument toward a peripheral end of the coronary artery. However, the tip end of the guiding catheter occasionally fails to stay in the ostium of the coronary artery if such indwelling is unstable with small backup force. In this case, an extension catheter having a small diameter is inserted to the guiding catheter to protrude from a distal opening of the guiding catheter, for increase in backup force.

There have been known various types of such extension catheters, including a guide extension catheter according to Patent Document 1, including a proximal member having an extension portion, a collar member attached to the extension portion, and a distal sheath member attached to the collar member. There has been further known a guide extension catheter according to Patent Document 2, including a push member including a segment having a first surface with a groove and a second surface opposite the first surface, and a distal shaft disposed adjacent to the push member and having a passageway. There has been also known a support catheter according to Patent Document 3, including a distal shaft constituting a distal part, and a proximal shaft constituting a proximal part and connected to the proximal part as the distal shaft with a modified polyolefin adhesive. Moreover, there have been known a coaxial guide catheter according to Patent Document 4, which is deliverable through a guide catheter with use of a guidewire rail segment, and a guide extension catheter according to Patent Document 5, including a distal sheath, a proximal shaft, and a coupling member fixing the distal sheath to the proximal shaft.

### RELATED ART DOCUMENTS

### Patent Documents

Patent Document 1: WO 2018/075700 A

Patent Document 2: WO 2017/214209 A

Patent Document 3: WO 2018/030075 A

Patent Document 4: U.S. Pat. No. 8,292,850

Patent Document 5: JP-A-2015-523186

### SUMMARY OF THE INVENTION

### Problems to be Solved by the Invention

An extension catheter is inserted to the vicinity of a stenosed part in an artery and is pulled back from the vicinity of the stenosed part, and is thus demanded to have sufficient breaking strength. There have been known various extension catheters with various efforts. There are further demands for improvement in breaking strength. The present invention has been achieved in view of the above problems, and it is an object of the present invention to provide an extension catheter having excellent breaking strength and a method of producing the same.

### Solutions to the Problems

The present invention provides an extension catheter and a method of producing the same as follows, which solve the above problems.

[1] An extension catheter including:
a tubular portion;
a tapered portion disposed proximally relative to the tubular portion; and
a linear member fixed to the tubular portion and the tapered portion;

the extension catheter satisfying Formula (1):

1.1T₂≤T₁≤6T₂ (1)
in which T₁ represents a thickness (mm) of the tubular portion at a position apart by 1 mm distally from a distal end of the tapered portion, the position being a position where the linear member is present, and T₂ represents a thickness (mm) of the tubular portion at a position apart by 1 mm distally from the distal end of the tapered portion, the position being opposite to the position where the linear member is present;

the extension catheter being configured to be inserted to a tubular catheter and protrude from a distal opening of the tubular catheter.

[2] The extension catheter according to above [1], wherein the linear member is composed of a metal.

[3] The extension catheter according to above [1] or [2], wherein

at the position apart by 1 mm distally from the distal end of the tapered portion,

the tubular portion has a part opposite to the position where the linear member is present, the part being composed of a resin.

[4] The extension catheter according to any one of above [1] to [3], further satisfying Formula (2):

1.1T₂≤T₁−T₃≤4T₂ (2)

in which T₁ and T₂ respectively represent the same thicknesses as the thicknesses represented by T₁ and T₂ in Formula (1), and T₃ represents a thickness (mm) of the linear member at the position apart by 1 mm distally from the distal end of the tapered portion.

[5] The extension catheter according to any one of above [1] to [4], further satisfying Formula (3):

1.1T₁≤T₅≤2T₁ (3)

in which T₁ represents the same thickness as the thickness represented by T₁ in Formula (1), and T₅ represents a thickness (mm) of the tapered portion at a position apart by 1 mm distally from a proximal end of the tapered portion, the position being a position where the linear member is present.

[6] The extension catheter according to any one of above [1] to [5], further satisfying Formula (4):

1.1T₆≤T₄≤2T₆ (4)

in which T₄ represents a minimum distance (mm) from the linear member to an inner surface of the tubular portion at the position apart by 1 mm distally from the distal end of the tapered portion, and T₆ represents a minimum distance (mm) from the linear member to an inner surface of the tapered portion at a position apart by 1 mm distally from a proximal end of the tapered portion.

[7] The extension catheter according to any one of above [1] to [6], further satisfying Formula (5):

1.1(T₅−T₇)≤T₁−T₃≤3(T₅−T₇) (5)

in which T₁ represents the same thickness as the thickness represented by T₁ in Formula (1), T₃ represents a thickness (mm) of the linear member at the position apart by 1 mm distally from the distal end of the tapered portion, T₅ represents a thickness (mm) of the tapered portion at a position apart by 1 mm distally from a proximal end of the tapered portion, the position being a position where the linear member is present, and T₇ represents a thickness (mm) of the linear member at the position apart by 1 mm distally from the proximal end of the tapered portion.

[8] The extension catheter according to any one of above [1] to [7], further including,

at the position apart by 1 mm distally from the distal end of the tapered portion,

a first resin disposed outside the linear member, and a second resin disposed still outside the first resin, the second resin having a higher melting point than a melting point of the first resin.

[9] The extension catheter according to any one of above [1] to [7], further including,

at the position apart by 1 mm distally from the distal end of the tapered portion,

a first resin disposed outside the linear member, and a second resin disposed still outside the first resin, the second resin having a higher Shore hardness than a Shore hardness of the first resin.

[10] The extension catheter according to any one of above [1] to [7], further including,

at the position apart by 1 mm distally from the distal end of the tapered portion,

a first resin disposed outside the linear member, and a third resin disposed at the position opposite to the position where the linear member is present, the third resin having a lower melting point than a melting point of the first resin.

[11] The extension catheter according to any one of above [1] to [7], further including,

at the position apart by 1 mm distally from the distal end of the tapered portion,

a first resin disposed outside the linear member, and a third resin disposed at the position opposite to the position where the linear member is present, the third resin having a lower Shore hardness than a Shore hardness of the first resin.

[12] The extension catheter according to any one of above [1] to [11], further satisfying Formula (6):

1.1T₉≤T₈≤6T₉ (6)

in which T₈ represents a thickness (mm) of a part of the tubular portion, the part being adjacent to the linear member and disposed at a position apart by 0.2 mm distally from a distal end of the linear member, and T₉ represents a thickness (mm) of a part of the tubular portion, the part being opposite to the linear member and disposed at the position apart by 0.2 mm distally from the distal end of the linear member.

[13] The extension catheter according to any one of above [1] to [12], further satisfying Formula (7):

1.1T₈≤T₅≤2T₈ (7)

in which T₈ represents a thickness (mm) of a part of the tubular portion, the part being adjacent to the linear member and disposed at a position apart by 0.2 mm distally from a distal end of the linear member, and T₅ represents a thickness (mm) of the tapered portion at a position apart by 1 mm distally from a proximal end of the tapered portion, the position being a position where the linear member is present.

[14] The extension catheter according to any one of above [1] to [13], wherein

T₁ is 0.1 mm or more and 0.4 mm or less, and

T₂ is 0.05 mm or more and 0.2 mm or less.

[15] A method of producing an extension catheter, the method including:

attaching a resin to a linear member and disposing the linear member on a tubular member;

surrounding the linear member and the tubular member with a heat-shrinkable film, shrinking the heat-shrinkable film by heat to fix the linear member to the tubular member; and

forming a tapered surface on the tubular member.

### Effects of the Invention

The present invention provides, in accordance with the above configurations, an extension catheter having excellent breaking strength and a method of producing the same.

### BRIEF DESCRIPTION OF THE DRAWINGS

FIG. 1 is a side view of an extension catheter according to an embodiment of the present invention.

FIG. 2 is an exemplary sectional view taken along line II-II in FIG. 1.

FIG. 3 is an exemplary sectional view taken along line III-III in FIG. 1.

FIG. 4 is another exemplary sectional view taken along line II-II in FIG. 1.

FIG. 5 is an exemplary sectional view taken along line III-III in FIG. 1.

FIG. 6 is a side view of a linear member.

FIG. 7 is still another exemplary sectional view taken along line II-II in FIG. 1.

FIG. 8 is further another exemplary sectional view taken along line II-II in FIG. 1.

FIG. 9 is an axial sectional view of a portion R in FIG. 1.

FIG. 10 is a sectional view taken along line X-X in FIG. 1.

FIG. 11 is a sectional view taken along line XI-XI in FIG. 1.

FIG. 12 is an exemplary sectional view taken along line III-III in FIG. 1.

FIG. 13 is a view of the extension catheter according to the embodiment of the present invention, in a state of being inserted to a tubular catheter and protruding from a distal opening of the tubular catheter.

### MODE FOR CARRYING OUT THE INVENTION

More specific description will now be made to the following embodiment of the present invention. The present invention should not be limited by the following embodiment but can obviously be implemented with appropriate modifications within a range applicable to purport of the above and below description. Such modifications will be all included in the technical scope of the present invention. Some of the drawings may not include reference signs of members for convenience, in which case the specification or any other appropriate drawing should be referred to. Various members in the drawings may not be depicted in actual sizes, because contribution to comprehension of the features of the present invention is prioritized in the drawings.

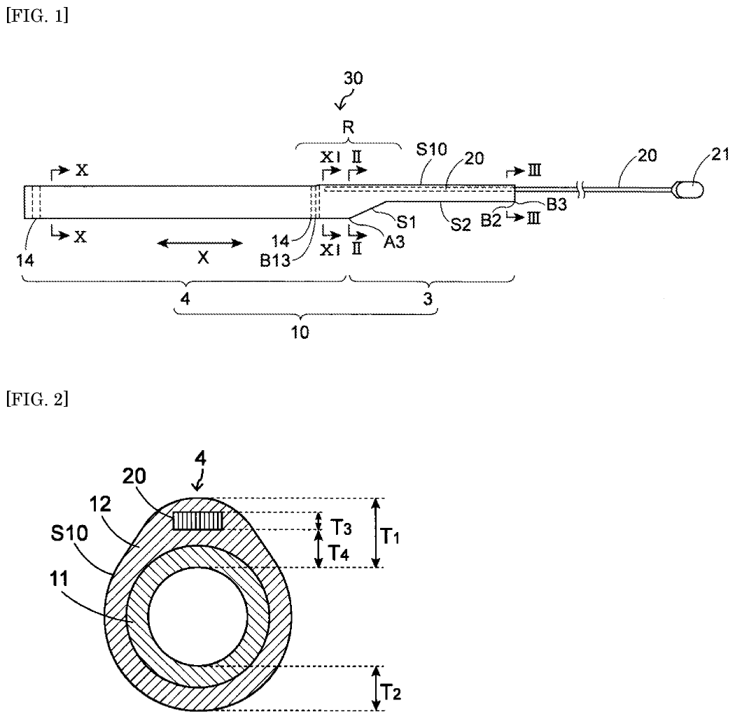

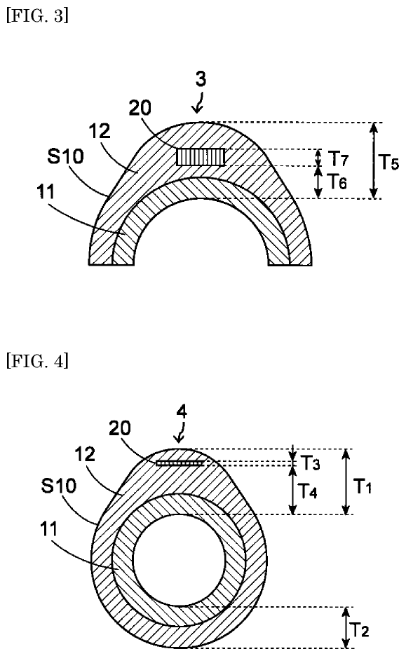

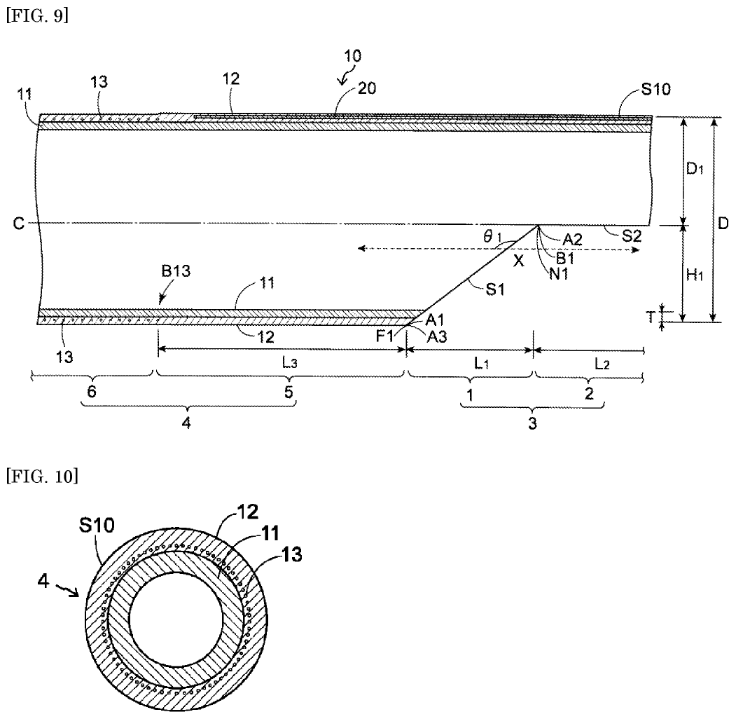

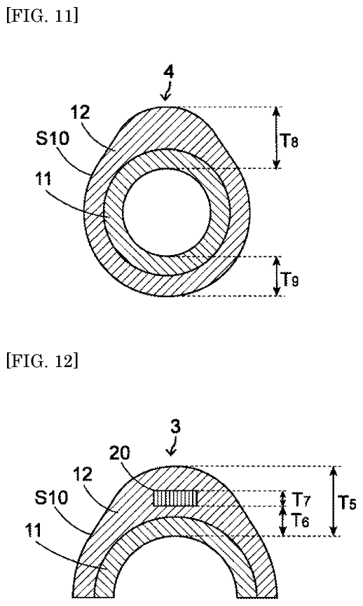

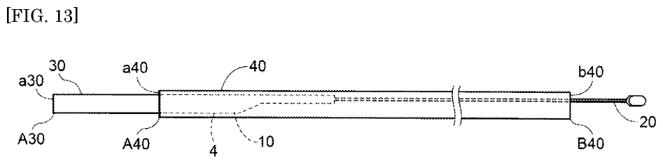

Description is made initially to an extension catheter according to the embodiment of the present invention with reference mainly to FIGS. 1 to 13. FIG. 1 is a side view of the extension catheter according to the embodiment of the present invention. FIG. 2 is an exemplary sectional view taken along line II-II in FIG. 1. FIGS. 3, 5, and 12 are exemplary sectional views taken along line III-III in FIG. 1. FIG. 6 is a side view of a linear member. FIG. 9 is an axial sectional view of a portion R in FIG. 1. FIG. 10 is a sectional view taken along line X-X in FIG. 1. FIG. 11 is a sectional view taken along line XI-XI in FIG. 1. FIGS. 4, 7, and 8 are other exemplary sectional views taken along line II-II in FIG. 1. FIG. 13 is a view of the extension catheter according to the embodiment of the present invention, in a state of being inserted to a tubular catheter and protruding from a distal opening of the tubular catheter.

FIG. 1 depicts an extension catheter 30 according to the embodiment of the present invention, and the extension catheter can be inserted to a tubular catheter 40 exemplarily depicted in FIG. 13 and can protrude from a distal opening a40 of the tubular catheter 40. Furthermore, as depicted in FIG. 1, the extension catheter 30 includes a tubular portion 4, a tapered portion 3 positioned proximally relative to the tubular portion 4, and a linear member 20 fixed to the tubular portion 4 and the tapered portion 3. Moreover, the extension catheter 30 is formed to satisfy Formula (1).

1.1T₂≤T₁≤6T₂ (1)

in which T₁ represents a thickness (mm) of the tubular portion 4 at a position apart by 1 mm distally from a distal end A3 of the tapered portion 3, the position being a position where the linear member 20 is present, and T₂ represents a thickness (mm) of the tubular portion 4 at a position apart by 1 mm distally from the distal end A3 of the tapered portion 3, the position being opposite to the position where the linear member 20 is present.

In the present invention, a proximal side indicates a side close to hands of an operator in an extending direction of the extension catheter 30, and a distal side indicates a side opposite the proximal side, that is, a side close to a treatment target.

The extension catheter 30 according to the embodiment of the present invention has the significant feature that the linear member 20 is fixed to the tapered portion 3 and the tubular portion 4, and that the tubular portion 4 has a part being adjacent to the linear member 20 and increased in thickness so as to satisfy Formula (1). Specifically, in some of conventional extension catheters according to Patent Documents 1, 3, and the like, a tubular distal member includes a tapered portion to form an opening, for easier insertion of an intravascular treatment instrument such as a stent or a balloon to the extension catheter. In such an extension catheter including the distal member having the tapered portion, a linear proximal member is fixed to the tapered portion. However, the tapered portion is easily deformed and the linear proximal member may thus be unfixed from the tapered portion, which may lead to breaking of the extension catheter. In contrast, the linear member 20 according to the present invention is fixed to the tubular portion 4 as well as to the tapered portion 3, and the tubular portion 4 has the part being adjacent to the linear member 20 and increased in thickness so as to satisfy Formula (1) for firm fixture of the linear member 20. The extension catheter 30 can thus be improved in breaking strength. The following description principally refers to formulae.

1.1T₂≤T₁≤6T₂ (1)

in which T₁ represents a thickness (mm) of the tubular portion 4 at a position apart by 1 mm distally from a distal end A3 of the tapered portion 3, the position being a position where the linear member 20 is present, and T₂ represents a thickness (mm) of the tubular portion 4 at a position apart by 1 mm distally from the distal end A3 of the tapered portion 3, the position being opposite to the position where the linear member 20 is present.

The position apart by 1 mm distally from the distal end A3 of the tapered portion 3 corresponds to a position on line II-II of the tubular portion 4 in FIG. 1, which may hereinafter be simply called a position II-II. FIG. 2 is a sectional view taken along line II-II in FIG. 1. As depicted in FIG. 2, on the position II-II, the thickness T₁ of the tubular portion 4 at the position where the linear member 20 is present is 1.1 times or more and six times or less of the thickness T₂ of the tubular portion 4 at the position opposite to the position where the linear member 20 is present. T₁ is 1.1 times T₂ or more, in other words, the thickness of the tubular portion 4 at the part adjacent to the linear member 20 is made larger than the thickness at the opposite part, for easier achievement of firm fixture of the linear member 20. Meanwhile, the thickness at the part opposite to the linear member 20 is decreased for easier improvement in flexibility at the opposite part. T₁ is preferably 1.3 times T₂ or more, is more preferably 1.5 times T₂ or more, and is further preferably 1.8 times T₂ or more. Meanwhile, T₁ is six times T₂ or less for easier reduction in outer diameter of the tubular portion 4. T₁ is preferably four times T₂ or less, is more preferably three times T₂ or less, and is further preferably 2.5 times T₂ or less.

Specifically, T₁ is preferably 0.02 mm or more and 0.5 mm or less, is more preferably 0.05 mm or more and 0.3 mm or less, and is further preferably 0.08 mm or more and 0.2 mm or less. Specifically, T₂ is preferably 0.01 mm or more and 0.3 mm or less, is more preferably 0.02 mm or more and 0.2 mm or less, and is further preferably 0.05 mm or more and 0.1 mm or less.

It is particularly preferred that T₁ is 0.1 mm or more and 0.4 mm or less and T₂ is 0.05 mm or more and 0.2 mm or less. The extension catheter thus easily achieves excellent operability and further improvement in breaking strength.

The extension catheter 30 is further preferred to satisfy Formula (2).

1.1T₂≤T₁−T₃≤4T₂ (2)

in which T₁ and T₂ respectively represent the same thicknesses as the thicknesses represented by T₁ and T₂ in Formula (1), and T₃ represents a thickness (mm) of the linear member 20 at the position apart by 1 mm distally from the distal end A3 of the tapered portion 3.

As depicted in FIG. 2, on the position II-II, a difference (T₁−T₃) between the thickness T₁ of the tubular portion 4 at the position where the linear member 20 is present and the thickness T₃ of the linear member 20 is preferably 1.1 times or more and four times or less of the thickness T₂ of the tubular portion 4 at the position opposite to the position where the linear member 20 is present. The difference (T₁−T₃) being 1.1 times T₂ or more easily achieves firm fixture of the linear member 20. The difference (T₁−T₃) is more preferably 1.3 times T₂ or more, is further preferably 1.5 times T₂ or more, and is still further preferably 1.8 times T₂ or more. Meanwhile, the difference (T₁−T₃) being four times T₂ or less easily achieves reduction in outer diameter of the tubular portion 4. The difference (T₁−T₃) is more preferably three times T₂ or less, and is further preferably 2.5 times T₂ or less.

Specifically, T₃ is preferably 0.001 mm or more and 0.3 mm or less, is more preferably 0.005 mm or more and 0.25 mm or less, and is further preferably 0.01 mm or more and 0.2 mm or less.

The extension catheter 30 is further preferred to satisfy Formula (3).

1.1T₁≤T₅≤2T₁ (3)

in which T₁ represents the same thickness as the thickness represented by T₁ in Formula (1), and T₅ represents a thickness (mm) of the tapered portion 3 at a position apart by 1 mm distally from a proximal end B3 of the tapered portion 3, the position being a position where the linear member 20 is present.

The position apart by 1 mm distally from the proximal end B3 of the tapered portion 3 corresponds to a position on line III-III in FIG. 1, which may hereinafter be simply called a position III-III. FIG. 3 is a sectional view taken along line III-III in FIG. 1. As depicted in FIGS. 2 and 3, the thickness T₅ of the tapered portion 3 at the position where the linear member 20 is present on the position III-III is preferably 1.1 times or more and two times or less of the thickness T₁ of the tubular portion 4 at the position where the linear member 20 is present on the position II-II. The tubular portion 4 can thus be easily pushed along the linear member 20. T₅ is more preferably 1.2 times T₁ or more, is further preferably 1.4 times T₁ or more, as well as is more preferably 1.8 times T₁ or less, and is further preferably 1.6 times T₁ or less.

Specifically, T₅ is preferably 0.03 mm or more and 0.6 mm or less, is more preferably 0.07 mm or more and 0.4 mm or less, and is further preferably 0.09 mm or more and 0.25 mm or less.

The extension catheter 30 is further preferred to satisfy Formula (4).

1.1T₆≤T₄≤2T₆ (4)

in which T₄ represents a minimum distance (mm) from the linear member 20 to an inner surface of the tubular portion 4 at the position apart by 1 mm distally from the distal end A3 of the tapered portion 3, and T₆ represents a minimum distance (mm) from the linear member 20 to an inner surface of the tapered portion 3 at the position apart by 1 mm distally from the proximal end B3 of the tapered portion 3.

As depicted in FIGS. 2 and 3, the minimum distance T₄ from the linear member 20 to the inner surface of the tubular portion 4 on the position II-II is preferably 1.1 times or more and two times or less of the minimum distance T₆ from the linear member 20 to the inner surface of the tapered portion 3 on the position III-III. The tubular portion 4 can thus be easily pushed along the linear member 20. T₄ is more preferably 1.2 times T₆ or more, is further preferably 1.4 times T₆ or more, as well as is more preferably 1.8 times T₆ or less, and is further preferably 1.6 times T₆ or less.

Specifically, T₄ is preferably 0.01 mm or more and 0.2 mm or less, is more preferably 0.02 mm or more and 0.1 mm or less, and is further preferably 0.03 mm or more and 0.07 mm or less. Specifically, T₆ is preferably 0.005 mm or more and 0.15 mm or less, is more preferably 0.01 mm or more and 0.08 mm or less, and is further preferably 0.02 mm or more and 0.05 mm or less.

The extension catheter 30 is preferred to satisfy Formula (5).

1.1(T₅−T₇)≤T₁−T₃≤3(T₅−T₇) (5)

in which T₁ represents the same thickness as the thickness represented by T₁ in Formula (1), T₃ represents a thickness (mm) of the linear member 20 at the position apart by 1 mm distally from the distal end A3 of the tapered portion 3, T₅ represents a thickness (mm) of the tapered portion 3 at the position apart by 1 mm distally from the proximal end B3 of the tapered portion 3, the position being a position where the linear member 20 is present, and T₇ represents a thickness (mm) of the linear member 20 at the position apart by 1 mm distally from the proximal end B3 of the tapered portion 3.

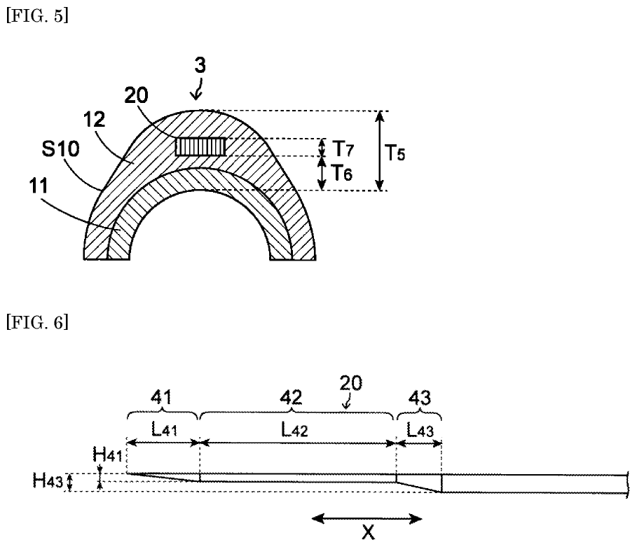

For example, the difference (T₁−T₃) between the thickness T₁ of the tubular portion 4 at the position where the linear member 20 is present and the thickness T₃ of the linear member 20 on the position II-II indicated in FIG. 4 is preferably 1.1 times or more and three times or less of a difference (T₅−T₇) between the thickness T₅ of the tapered portion 3 at the position where the linear member 20 is present and the thickness T₇ of the linear member 20 on the position III-III indicated in FIG. 5. The linear member 20 can thus be easily balanced in rigidity, and the tubular portion 4 can thus be easily pushed along the linear member 20. The difference (T₁−T₃) is more preferably 1.3 times (T₅−T₇) or more, is further preferably 1.5 times (T₅−T₇) or more, as well as is more preferably 2.5 times (T₅−T₇) or less, and is further preferably two times (T₅−T₇) or less.

Specifically, T₇ is preferably 0.005 mm or more and 0.35 mm or less, is more preferably 0.01 mm or more and 0.3 mm or less, and is further preferably 0.15 mm or more and 0.25 mm or less.

The extension catheter 30 is further preferred to satisfy Formula (6).

1.1T₉≤T₈≤6T₉ (6)

in which T₈ represents a thickness (mm) of a part of the tubular portion 4, the part being adjacent to the linear member 20 and disposed at a position apart by 0.2 mm distally from a distal end of the linear member 20, and T₉ represents a thickness (mm) of a part of the tubular portion 4, the part being opposite to the linear member 20 and disposed at the position apart by 0.2 mm distally from the distal end of the linear member 20.

The position apart by 0.2 mm distally from the distal end of the linear member 20 corresponds to a position on line XI-XI of the tubular portion 4 in FIG. 1, which may hereinafter be simply called a position XI-XI. FIG. 11 is a sectional view taken along line XI-XI in FIG. 1. As depicted in FIG. 11, on the position XI-XI, the thickness T₈ of the tubular portion 4 at the part adjacent to the linear member 20 is preferably 1.1 times or more and six times or less of the thickness T₉ of the tubular portion 4 at the part opposite to the linear member 20. T₈ is 1.1 times T₉ or more, in other words, the thickness of the tubular portion 4 at the part adjacent to the linear member 20 is made larger than the thickness at the opposite part, for easier achievement of firm fixture in the vicinity of the distal end of the linear member 20. Meanwhile, the thickness at the part opposite to the linear member 20 is decreased for easier improvement in flexibility at the opposite part. T₈ is preferably 1.3 times T₉ or more, is more preferably 1.5 times T₉ or more, and is further preferably 1.8 times T₉ or more. Meanwhile, T₈ is six times T₉ or less for easier reduction in outer diameter of the tubular portion 4. T₈ is preferably four times T₉ or less, is more preferably three times T₉ or less, and is further preferably 2.5 times T₉ or less.

Specifically, T₈ is preferably 0.02 mm or more and 0.5 mm or less, is more preferably 0.05 mm or more and 0.3 mm or less, and is further preferably 0.08 mm or more and 0.2 mm or less. Specifically, T₉ is preferably 0.01 mm or more and 0.3 mm or less, is more preferably 0.02 mm or more and 0.2 mm or less, and is further preferably 0.05 mm or more and 0.1 mm or less.

The extension catheter 30 is further preferred to satisfy Formula (7).

1.1T₈≤T₅≤2T₈ (7)

in which T₈ represents a thickness (mm) of a part of the tubular portion 4, the part being adjacent to the linear member 20 and disposed at a position apart by 0.2 mm distally from a distal end of the linear member 20, and T₅ represents a thickness (mm) of the tapered portion 3 at a position apart by 1 mm distally from the proximal end of the tapered portion 3, the position being a position where the linear member 20 is present.

FIG. 11 is a sectional view taken along line XI-XI in FIG. 1, and FIG. 12 is an exemplary sectional view taken along line III-III in FIG. 1. As depicted in FIGS. 11 and 12, the thickness T₅ of the tapered portion 3 at the position adjacent to the position where the linear member 20 is present on the position III-III is preferably 1.1 times or more and two times or less of the thickness T₈ of the tubular portion 4 at the part adjacent to the linear member 20 on the position XI-XI. The tubular portion 4 can thus be easily pushed along the linear member 20. T₅ is more preferably 1.2 times T₈ or more, is further preferably 1.4 times T₈ or more, as well as is more preferably 1.8 times T₈ or less, and is further preferably 1.6 times T₈ or less.

Furthermore, the extension catheter 30 includes a reinforcing layer 13 to be described later and being disposed at the tubular portion 4, and is preferred to satisfy Formula (8).

D≤L₃≤6D (8)

in which L₃ represents a length (mm) in an axial direction X of the tubular portion 4 from a proximal end B13 of the reinforcing layer 13 to the distal end A3 of the tapered portion 3, and D represents an outer diameter (mm) of the tubular portion 4 at the distal end A3 of the tapered portion 3.

As depicted in FIG. 9(a), the length L₃ in the axial direction X of the tubular portion 4 from the proximal end B13 of the reinforcing layer 13 to the distal end A3 of the tapered portion 3 is preferably equal to or more than the outer diameter D of the tubular portion 4 at the distal end A3 of the tapered portion 3. L₃ corresponds to a length, in the axial direction X of the tubular portion 4, of an unreinforced portion 5 where the reinforcing layer 13 is not present in the tubular portion 4. L₃ equal to or more than D will easily prevent deformation of the tapered portion 3. L₃ is preferably 1.5 times D or more, and is more preferably two times D or more. Meanwhile, L₃ is six times D or less for easier prevention of deformation of the unreinforced portion 5. L₃ is more preferably four times D or less, and is further preferably three times D or less.

Specifically, L₃ is preferably 0.5 mm or more and 8 mm or less, is more preferably 2 mm or more and 6 mm or less, and is further preferably 3 mm or more and 5 mm or less.

At the position apart by 1 mm distally from the distal end A3 of the tapered portion (on the position II-II), the tubular portion 4 has a part opposite to the position where the linear member 20 is present, and the part is preferably composed of a resin. This improves flexibility of the tubular portion 4 at the part opposite to the position where the linear member 20 is present. The tubular portion 4 may contain a radiopaque substance or the like to be described later within a range not impairing flexibility.

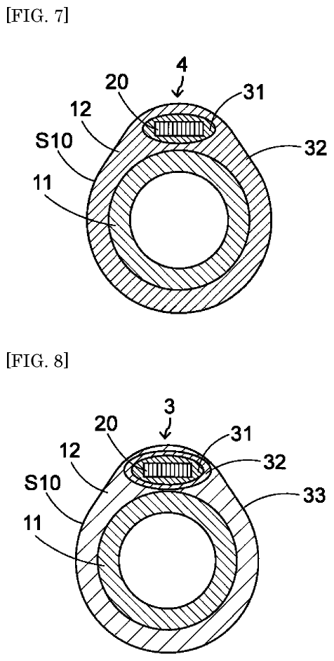

FIG. 7 is still another exemplary sectional view taken along line II-II in FIG. 1. As depicted in FIG. 7, at the position apart by 1 mm distally from the distal end A3 of the tapered portion 3 (on the position II-II), preferably, a first resin 31 is disposed outside the linear member 20, and a second resin 32 is disposed still outside the first resin 31, the second resin 32 having a higher melting point than a melting point of the first resin 31. The linear member 20 is bonded to the tubular portion 4 by the first resin 31 having the low melting point and is covered with the second resin 32 having the higher melting point, for easier achievement of firm fixture of the linear member 20. Though not depicted, also on the position III-III, preferably, the first resin 31 is disposed outside the linear member 20, and the second resin 32 is disposed still outside the first resin 31, the second resin 32 having the higher melting point than the melting point of the first resin 31.

As depicted in FIG. 7, at the position apart by 1 mm distally from the distal end A3 of the tapered portion 3 (on the position II-II), preferably, the first resin 31 is disposed outside the linear member 20, and the second resin 32 is disposed still outside the first resin 31, the second resin 32 having a higher Shore hardness than a Shore hardness of the first resin 31. This will easily prevent extension of cracking caused by curving, bending, or the like. Though not depicted, also on the position III-III, preferably, the first resin 31 is disposed outside the linear member 20, and the second resin 32 is disposed still outside the first resin 31, the second resin 32 having the higher Shore hardness than the Shore hardness of the first resin 31.

The Shore hardness can be measured in accordance with the ISO868: 2003 plastic durometer hardness test method using a type D durometer.

FIG. 8 is further another exemplary sectional view taken along line II-II in FIG. 1. As depicted in FIG. 8, at the position apart by 1 mm distally from the distal end A3 of the tapered portion 3 (on the position II-II), preferably, the first resin 31 is disposed outside the linear member 20, and a third resin 33 is further disposed at the position opposite to the position where the linear member 20 is present, the third resin 33 having a lower melting point than the melting point of the first resin 31. This easily improves flexibility at the part opposite to the linear member 20.

As depicted in FIG. 8, at the position apart by 1 mm distally from the distal end A3 of the tapered portion 3 (on the position II-II), preferably, the first resin 31 is disposed outside the linear member 20, and the third resin 33 is further disposed at the position opposite to the position where the linear member 20 is present, the third resin 33 having a lower Shore hardness than the Shore hardness of the first resin 31. This easily improves flexibility at the part opposite to the linear member 20.

Examples of the first resin 31, the second resin 32, and the third resin 33 include a resin as a material for an outer layer 12 to be described later.

Members of the extension catheter 30 will be described in detail next. As depicted in FIG. 1, the extension catheter 30 includes the tubular portion 4, the tapered portion 3 positioned proximally relative to the tubular portion 4, and the linear member 20 fixed to the tubular portion 4 and the tapered portion 3.

The extension catheter 30 is inserted to the tubular catheter 40 depicted in FIG. 13 or the like through a proximal opening b40 of the tubular catheter 40. The tubular catheter 40 has a distal end A40 having the opening a40 and a proximal end B40 having the opening b40. Examples of the tubular catheter 40 include a guiding catheter. The extension catheter 30 has a distal end A30 that is inserted through the proximal opening b40 of the tubular catheter 40. As depicted in FIG. 13, a distal portion of the extension catheter 30 can protrude from the distal opening a40 of the tubular catheter 40 and can be pulled backward. The extension catheter 30 is inserted to the tubular catheter 40 previously disposed in a body cavity, and is used to allow a device passing through the tubular catheter 40 and the extension catheter 30 to reach a further distal position in the body cavity. The tubular catheter 40 has an inner diameter larger than an outer diameter of the extension catheter 30 so as to receive the extension catheter 30 in a lumen of the tubular catheter 40. As depicted in FIG. 1, the extension catheter 30 is preferably shaped by coupling a distal member 10 including the tubular portion 4 and the tapered portion 3 disposed on the distal side and the linear member 20 having a bar shape. As depicted in FIG. 13, the linear member 20 of the extension catheter 30 is preferably used without protruding from the distal opening a40 of the tubular catheter 40. The extension catheter 30 can be exemplarily 1500 mm long, and the distal member 10 of the extension catheter 30 can be exemplarily 350 mm long. The distal member 10 of the extension catheter 30 has a distal end diameter exemplarily having 1.5 mm. When the extension catheter 30 is used, a treatment device can protrude from an opening a30 at the distal end A30 of the extension catheter through the tubular catheter 40 and the extension catheter 30. The treatment device can enter the tubular catheter 40 through the proximal opening b40 of the tubular catheter 40, can enter the extension catheter 30 through a proximal opening of the extension catheter 30, and can protrude from the distal opening a30 of the extension catheter 30.

The tubular portion 4 has an inner diameter preferably 1.0 mm or more and 2.2 mm or less. When the inner diameter of the tubular portion 4 is 2.2 mm or less, the tapered portion 3 is less likely to be deformed. The inner diameter is more preferably 2.0 mm or less, and is further preferably 1.8 mm or less. Meanwhile, the inner diameter of the tubular portion 4 is set to 1.0 mm or more for easier passage of an intravascular treatment instrument through the tubular portion 4. The inner diameter is more preferably 1.2 mm or more, and is further preferably 1.4 mm or more.

The outer diameter of the tubular portion 4 is preferably 1.2 mm or more and 3 mm or less. When the outer diameter of the tubular portion 4 is 3 mm or less, the tubular portion 4 can be easily inserted to a guiding catheter or a blood vessel. The outer diameter is more preferably 2 mm or less, and is further preferably 1.8 mm or less. Meanwhile, the outer diameter of the tubular portion 4 is set to 1.2 mm or more for easier improvement in strength of the tubular portion 4. The outer diameter is more preferably 1.4 mm or more, and is further preferably 1.6 mm or more.

As depicted in FIGS. 2, 9, and 10, the tubular portion 4 preferably includes an inner layer 11. The inner layer 11 is composed of a material such as a resin. The resin preferably includes at least one selected from the group consisting of a polyamide resin, a polyester resin, a polyurethane resin, a polyolefin resin, a fluororesin, a vinyl chloride resin, a silicone resin, and natural rubber. The resin more preferably includes at least one selected from the group consisting of the polyester resin, the polyolefin resin, the fluororesin, the silicone resin, and the natural rubber. The fluororesin is particularly preferred due to excellent chemical resistance, excellent nonadhesiveness, and excellent low friction.

Examples of the polyamide resin include nylon 12, nylon 12 elastomer, nylon 6, and an aromatic polyamide. Examples of the polyester resin include polyethylene terephthalate. Examples of the polyurethane resin include aliphatic polyurethane containing aliphatic isocyanate as a monomeric unit, and aromatic polyurethane containing aromatic isocyanate as a monomeric unit. Examples of the polyolefin resin include polyethylene and polypropylene. Examples of the fluororesin include polytetrafluoroethylene, ethylene tetrafluoroethylene, and fluorinated ethylene propylene. Polytetrafluoroethylene is preferably high strength polytetrafluoroethylene. Examples of the vinyl chloride resin include polyvinyl chloride and polyvinylidene chloride. Examples of the silicone resin include dimethylpolysiloxane, methylphenylpolysiloxane, methyl vinyl polysiloxane, and fluoroalkyl methylpolysiloxane. Examples of the natural rubber include latex.

The inner layer 11 may partially or entirely contain a radiopaque substance to be described later for easier recognition of the position of the tubular portion 4 by X-ray fluoroscopy or the like.

The tubular portion 4 preferably includes the outer layer 12. Examples of the material composing the outer layer 12 include a resin. The resin preferably includes at least one selected from the group consisting of a polyamide resin, a polyester resin, a polyurethane resin, a polyolefin resin, a fluororesin, a vinyl chloride resin, a silicone resin, and natural rubber. Among these, the resin more preferably includes at least one selected from the group consisting of the polyamide resin, the polyurethane resin, and the polyolefin resin, further preferably includes at least one selected from the group consisting of the polyamide resin and the polyurethane resin, and still further preferably includes the polyurethane resin.

The outer layer 12 may partially or entirely contain a radiopaque substance for easier recognition of the position of the tubular portion 4 by X-ray fluoroscopy or the like. Examples of the radiopaque substance include lead, barium, iodine, tungsten, aurum, platinum, iridium, a platinum-iridium alloy, stainless steel, titanium, a cobalt-chromium alloy, palladium, and tantalum.

The outer layer 12 has an outer surface preferably coated with a hydrophilic polymer. The tubular portion 4 can thus be easily inserted to a guiding catheter or a blood vessel. Examples of the hydrophilic polymer include poly 2-hydroxyethyl methacrylate, polyacrylamide, polyvinylpyrrolidone, a maleic anhydride copolymer such as a methyl vinyl ether maleic anhydride copolymer.

As depicted in FIGS. 9 and, 10, the tubular portion 4 preferably includes the reinforcing layer 13. The reinforcing layer 13 improves rigidity of the tubular portion 4. The reinforcing layer 13 is not limitedly disposed in the outer layer 12, but may alternatively be disposed in the inner layer 11 or between the inner layer 11 and the outer layer 12. In particular, the reinforcing layer 13 disposed in the outer layer 12 easily improves strength. The reinforcing layer 13 is thus preferably disposed in the outer layer 12.

Examples of the material composing the reinforcing layer 13 include metal wire, and fiber. Examples of the material composing the metal wire preferably include stainless steel, titanium, a nickel-titanium alloy, a cobalt-chromium alloy, and a tungsten alloy. Among these, the stainless steel is more preferred. The metal wire may include a single wire or a twisted wire. Examples of the fiber include polyarylate fiber, aramid fiber, ultrahigh molecular weight polyethylene fiber, PBO fiber, and carbon fiber. The fiber may be a monofilament or a multifilament.

The reinforcing layer 13 is not particularly limited in shape, but preferably has a spiral shape, a reticulated shape, or a braided shape. Among these shapes, the braided shape easily achieves improvement in rigidity. The reinforcing layer 13 thus more preferably has such a braided shape.

The reinforcing layer 13 may contain the radiopaque substance for easier recognition of the position of the tubular portion 4 by X-ray fluoroscopy or the like.

As depicted in FIG. 1, the tubular portion 4 has a tip portion preferably having a radiopaque marker 14. Specifically, the radiopaque marker 14 is preferably disposed at a site apart from a distal end of the tubular portion 4 by 50 mm or less as a distance in the axial direction X of the tubular portion 4, is more preferably disposed at a site apart by 20 mm or less, and is further preferably disposed at a site apart by 5 mm or less. This facilitates recognition of the position of the distal end of the tubular portion 4 in an artery.

As depicted in FIG. 1, the tubular portion 4 has a proximal part preferably having a radiopaque marker 14. Specifically, the radiopaque marker 14 is preferably disposed at a site apart from the proximal end B13 of the reinforcing layer 13 by 50 mm or less as a distance in the axial direction X of the tubular portion 4. The radiopaque marker 14 is more preferably disposed at a site apart by 20 mm or less from the proximal end B13 of the reinforcing layer 13, and is further preferably disposed at a site apart by 5 mm or less from the proximal end B13 of the reinforcing layer 13. This facilitates recognition of the position of a proximal part of the tubular portion 4 in an artery.

The radiopaque marker 14 is not particularly limited in shape, and examples of the shape include a belt shape and a spiral shape. The examples of the material composing radiopaque marker 14 include the radiopaque substance.

The tapered portion 3 corresponds to an opening for insertion of an intravascular treatment instrument such as a stent or a balloon to the extension catheter. The tapered portion 3 has an outer surface S10 and a tapered surface.

The extension catheter 30 may include a plurality of tapered portions. As depicted in FIG. 9, the extension catheter 30 preferably includes a first tapered portion 1 positioned proximally relative to the tubular portion 4, and a second tapered portion 2 positioned proximally relative to the first tapered portion 1.

The first tapered portion 1 and the second tapered portion 2 have a first tapered surface S1 and a second tapered surface S2, respectively. The first tapered portion 1 and the second tapered portion 2 each have the outer surface S10. The outer surfaces S10 of the tapered portions have a radial sectional shape preferably deformed gradually from a circular shape as depicted in FIG. 2 to an arc shape as depicted in FIG. 3 from the first tapered portion 1 toward the second tapered portion 2.

The first tapered portion 1 is preferred to satisfy Formula (9).

90°≤θ₁≤145° (9)

in which θ₁ represents an angle between the first tapered surface S1 and the axial direction X of the tubular portion 4.

As depicted in FIG. 9, the angle θ₁ between the first tapered surface S1 and the axial direction X of the tubular portion 4 is preferably 90 degrees or more and 145 degrees or less. θ₁ set to 145 degrees or less achieves reduction of a thin part of the tubular portion 4 at the distal end A3 of the tapered portion 3 and an intravascular treatment instrument is thus less likely to be caught. θ₁ is thus more preferably 140 degrees or less, is further preferably 130 degrees or less, and is still further preferably 120 degrees or less. Meanwhile, θ₁ set to 90 degrees or more facilitates increase in opening area of the opening for insertion of an intravascular treatment instrument. θ₁ is thus more preferably 95 degrees or more, is further preferably 100 degrees or more, and is still further preferably 110 degrees or more.

The extension catheter 30 is further preferred to satisfy Formula (10).

0.3D≤D₁ (10)

in which D represents an outer diameter (mm) of the tubular portion 4 at a distal end A1 of the first tapered portion 1, and D₁ represents a length in the radial direction at a proximal end B1 of the first tapered portion 1.

The length D₁ in the radial direction at the proximal end B1 of the first tapered portion 1 is preferably 0.3 times or more of the outer diameter D of the tubular portion 4 at the distal end A1 of the first tapered portion 1. This easily prevents local bending in the vicinity of the proximal end B1 of the first tapered portion 1. D₁ is more preferably 0.4 times D or more. Meanwhile, D₁ is preferably 0.8 times D or less. The opening is thus enlarged for easier insertion of an intravascular treatment instrument. Accordingly, D₁ is more preferably 0.7 times D or less, and is further preferably 0.6 times D or less.

Specifically, D₁ is preferably 0.2 mm or more and 2 mm or less, is more preferably 0.5 mm or more and 1.5 mm or less, and is further preferably 0.8 mm or more and 1.2 mm or less. D is preferably 0.5 mm or more and 4 mm or less, is more preferably 1 mm or more and 2 mm or less, and is further preferably 1.4 mm or more and 1.8 mm or less.

A length L₁ in the axial direction X from a farthest point F1 on the first tapered surface S1 from a central axis C of the tubular portion 4 to a closest point N1 on the first tapered surface S1 to the central axis C of the tubular portion 4 is preferably 0.5 mm or more and 6 mm or less, is more preferably 1 mm or more and 4 mm or less, and is further preferably 1.5 mm or more and 3 mm or less.

A length H₁ in the radial direction between the closest point N1 and the farthest point F1 on the first tapered surface S1 from the central axis C of the tubular portion 4 is preferably 0.1 mm or more and 1.5 mm or less, is more preferably 0.2 mm or more and 1 mm or less, and is further preferably 0.4 mm or more and 0.7 mm or less.

Furthermore, the extension catheter 30 preferably includes the second tapered portion 2 positioned proximally relative to the first tapered portion 1. The second tapered portion 2 has the outer surface S10 and the second tapered surface S2. The second tapered portion 2 is disposed for easier insertion of an intravascular treatment instrument to the opening along the second tapered portion 2.

The extension catheter 30 is further preferred to satisfy Formula (11).

−5°≤θ₂≤5° (11)

in which θ₂ represents an angle between the second tapered surface S2 and the axial direction X of the tubular portion 4.

As in Formula (11), the angle θ₂ (not indicated) between the second tapered surface S2 and the axial direction X of the tubular portion 4 is set to −5 degrees or more and 5 degrees or less, that is, the second tapered surface S2 and the axial direction X of the tubular portion 4 are substantially in parallel with each other, for easier insertion of an intravascular treatment instrument to the opening along the second tapered portion 2. The angle θ₂ is more preferably −3 degrees or more and 3 degrees or less, is further preferably −2 degrees or more and 2 degrees or less, and is still further preferably −1 degree or more and 1 degree or less.

The extension catheter 30 is further preferred to satisfy Formula (12).

10D≤L₂≤200D (12)

in which D represents an outer diameter (mm) of the tubular portion 4 at a distal end A1 of the first tapered portion 1, and L₂ represents a length (mm) in the axial direction X of the tubular portion 4 from a distal end of the second tapered portion 2 to a proximal end B2 of the second tapered portion 2.

The length L₂ in the axial direction X of the tubular portion 4 from a distal end A2 of the second tapered portion 2 to the proximal end B2 of the second tapered portion 2 is preferably ten times or more of the outer diameter D of the tubular portion 4 at the distal end A1 of the first tapered portion 1. This easily increases a contact area between the second tapered portion 2 and the linear member 20, and the linear member 20 is easily fixed firmly. L₂ is more preferably 30 times the outer diameter D or more, and is further preferably 60 times the outer diameter D or more. Meanwhile, L₂ is preferably 200 times the outer diameter D or less. This improves flexibility of the linear member 20. L₂ is more preferably 120 times the outer diameter D or less, and is further preferably 90 times the outer diameter D or less.

Specifically, L₂ is preferably 5 cm or more and 20 cm or less, is more preferably 10 cm or more and 18 cm or less, and is further preferably 12 cm or more and 15 cm or less.

A length in the axial direction X of the tubular portion 4 from the distal end (not depicted) to the proximal end (not depicted) of the tubular portion 4 is preferably 10 cm or more and 50 cm or less, is more preferably 20 cm or more and 40 cm or less, and is further preferably 25 cm or more and 35 cm or less.

As depicted in FIGS. 1 and 9, the first tapered surface S1 and the second tapered surface S2 are preferably disposed adjacent to each other. There may be optionally disposed a third tapered surface (not depicted) between the first tapered surface S1 and the second tapered surface S2. In this case, the first tapered surface S1 and the third tapered surface are preferably disposed adjacent to each other. Furthermore, the third tapered surface and the second tapered surface S2 are preferably disposed adjacent to each other.

An angle θ₃ (not indicated) between the third tapered surface and the axial direction X of the tubular portion 4 is preferably 120 degrees or more. This facilitates increase in opening area of the opening for insertion of an intravascular treatment instrument. θ₃ is thus more preferably 130 degrees or more, is further preferably 140 degrees or more, and is still further preferably 150 degrees or more. Meanwhile, θ₃ has an upper limit that may be 175 degrees, 170 degrees, 168 degrees, or the like.

A length in the axial direction X from a farthest point to a closest point on the third tapered surface from and to the central axis C of the tubular portion 4 is preferably 0.5 mm or more and 6 mm or less, is more preferably 1 mm or more and 4 mm or less, and is further preferably 1.5 mm or more and 3 mm or less.

A length in the radial direction between the closest point and the farthest point on the third tapered surface from the central axis C of the tubular portion 4 is preferably 0.1 mm or more and 1.5 mm or less, is more preferably 0.2 mm or more and 1 mm or less, and is further preferably 0.4 mm or more and 0.7 mm or less.

Each of the first tapered portion 1 and the second tapered portion 2 preferably includes an inner layer 11. The inner layers 11 are each composed of a material exemplified as the material for the inner layer 11 of the tubular portion 4. The first tapered portion 1 and the second tapered portion 2 are each composed of a material preferably the same as the material for the inner layer 11 of the tubular portion 4.

Each of the first tapered portion 1 and the second tapered portion 2 preferably includes an outer layer 12. The outer layers 12 are each composed of a material exemplified as the material for the outer layer 12 of the tubular portion 4. The material for each of the outer layers 12 of the first tapered portion 1 and the second tapered portion 2 may be the same as or different from the material for the outer layer 12 of the tubular portion 4, but the outer layers 12 of the first tapered portion 1 and the second tapered portion 2 preferably include a common resin. This easily prevents local bending of the tapered portions.

Each of the outer layer 12 of the first tapered portion 1 and the outer layer 12 of the second tapered portion 2 preferably contains a resin having a higher Shore hardness than a Shore hardness of a resin contained in a reinforced portion 6, where the reinforcing layer 13 is present, in the outer layer 12 of the tubular portion 4. This secures flexibility of the tubular portion 4 as well as easily prevents deformation of the opening.

The linear member 20 is a long wire rod. The linear member 20 pushes the tubular portion 4 to cause the tubular portion 4 to protrude from the opening of the tubular catheter (not depicted).

As depicted in FIG. 9, the linear member 20 is preferably fixed to the outer layer 12 of the unreinforced portion 5 and the outer layer 12 of the tapered portion 3. The linear member 20 can thus be easily fixed firmly to the distal member 10 including the tubular portion 4 and the tapered portion 3. The linear member 20 is more preferably fixed to the outer layer 12 of the reinforced portion 6, the outer layer 12 of the unreinforced portion 5, and the outer layer 12 of the tapered portion 3.

The linear member 20 is preferably composed of a metal. Specifically, the linear member 20 is preferably composed of a material such as stainless steel, titanium, a nickel-titanium alloy, and a cobalt-chromium alloy, a tungsten alloy, or the like. Among these, the stainless steel is more preferred. The linear member 20 has a sectional shape in a thickness direction not limited to the shape depicted in each of FIGS. 2 and 3, but may alternatively have a square shape, a rectangular shape, a trapezoidal shape, a circular shape, or the like. The rectangular shape is preferably selected among these.

The linear member 20 may optionally have a tapered portion in a side view as depicted in FIG. 6. The linear member 20 having such a tapered portion facilitates control of the thickness so as to satisfy Formula (4) and Formula (5). Furthermore, the linear member 20 can be easily balanced in rigidity, and the tubular portion 4 can thus be easily pushed along the linear member 20.

The linear member 20 depicted in FIG. 6 has a distal part including a first tapered portion 41. The first tapered portion 41 is preferably fixed to the tubular portion 4 and the tapered portion 3. The first tapered portion 41 has an axial length L₄₁ preferably 5 mm or more and 25 mm or less and more preferably 10 mm or more and 20 mm or less. The first tapered portion 41 has a proximal end having a thickness H₄₁ preferably 0.1 mm or more and 0.5 mm or less and more preferably 0.2 mm or more and 0.4 mm or less. The first tapered portion 41 has a distal end having a thickness preferably 0.02 mm or more and 0.2 mm or less and more preferably 0.05 mm or more and 0.13 mm or less.

As depicted in FIG. 6, the linear member 20 preferably includes a second tapered portion 42. The second tapered portion 42 has an axial length L₄₂ preferably five times or more and twelve times or less of L₄₁, and more preferably seven times or more and ten times or less of L₄₁. The second tapered portion 42 has a thickness preferably 0.8 times or more and 1.2 times or less of H₄₁, and more preferably 0.9 times or more and 1.1 times or less of H₄₁. The second tapered portion 42 is preferably fixed to the tapered portion 3.

Furthermore, as depicted in FIG. 6, the linear member 20 preferably includes a third tapered portion 43. The third tapered portion 43 has an axial length L₄₃ preferably 0.1 times or more and 0.6 times or less of L₄₁, and more preferably 0.2 times or more and 0.4 times or less of L₄₁. The third tapered portion 43 has a proximal end having a thickness H₄₃ preferably 0.15 times or more and 0.5 times or less of H₄₁, and more preferably 0.2 times or more and 0.4 times or less of H₄₁.

As depicted in FIG. 1, the linear member 20 has a proximal part preferably having a grip member 21. The grip member 21 is attached to a proximal end of the linear member 20 and is shaped to be gripped with fingers of an operator. The grip member 21 is composed of a material such as a resin, and examples of the resin include a polyolefin resin such as polyethylene or polypropylene.

Description is next made to a method of producing the extension catheter 30 according to the embodiment of the present invention. Examples of the method of producing the extension catheter 30 include a production method including: attaching a resin to the linear member 20 and disposing the linear member 20 on a tubular member; surrounding the linear member 20 and the tubular member with a heat-shrinkable film and shrinking the heat-shrinkable film by heat to fix the linear member 20 to the tubular member; and forming a tapered surface on the tubular member.

Attaching the resin to the linear member 20 preferably includes attaching the first resin 31. In a case where the linear member 20 and the first resin 31 are heated to bury the linear member 20 in the outer layer 12 composed of the second resin 32, the first resin 31 can be disposed outside the linear member 20 and the second resin 32 can be disposed still outside the first resin 31 as depicted in FIG. 7.

Attaching the resin to the linear member 20 preferably includes attaching the first resin 31 and attaching the second resin 32 to be disposed still outside the first resin 31. When the second resin 32 is heated to fix the linear member 20 in the outer layer 12 composed of the second resin 32, the second resin 32 same as the material for the outer layer 12 is preliminarily attached to the linear member 20 for easier fitting between the resin attached to the linear member 20 and the resin of the outer layer 12. Also in this case, the first resin 31 can be disposed outside the linear member 20 and the second resin 32 can be disposed still outside the first resin 31 as depicted in FIG. 7. When the first resin 31 and the second resin 32 are attached to the linear member 20, the linear member 20 may be covered with a tube including a first layer containing the first resin 31 and a second layer containing the second resin 32.

When the resin is attached to the linear member 20, the resin may be changed in thickness in the axial direction X of the tubular portion 4, or the resin may be changed in thickness at sites above and below the linear member 20. This facilitates control so as to satisfy Formula (3) to Formula (5).

The linear member 20 and the tubular member are surrounded with the heat-shrinkable film that is shrunk by heat to fix linear member 20 to the tubular member, for easier fixture of the linear member 20 in the outer layer 12. When the heat-shrinkable film is used, preferably, a metal core is inserted to the tubular member and after that, the linear member 20 and the tubular member are surrounded with the heat-shrinkable film to be shrunk by heat.

Forming the tapered surface on the tubular member includes forming the first tapered surface S1. Forming the first tapered surface S1 preferably includes cutting at an angle satisfying Formula (9). Furthermore, forming the first tapered surface S1 enables easy control of an angle of cutting in the second tapered surface S2.

Moreover, forming the tapered surface on the tubular member preferably includes forming the second tapered surface S2. Forming the second tapered surface S2 preferably includes cutting at an angle satisfying Formula (11).

In a case where forming the second tapered surface S2 includes cutting from a position proximally relative to the first tapered surface S1 toward a distal side to form the second tapered surface S2, the second tapered surface S2 can be easily formed planarly. Optionally, the first tapered surface S1 may be cut from a distal end of the first tapered surface S1 toward a proximal side. In this case, the first tapered surface S1 and the second tapered surface S2 can be formed through successive cutting.

There may be optionally formed a tapered surface other than the first tapered surface S1 and the second tapered surface S2. Such cutting may be made with use of a cutting tool such as a cutter.

Forming the tapered surface on the tubular member achieves the extension catheter 30 including the distal member 10 having the tubular portion 4 and the tapered portion 3, and the linear member 20 fixed to the distal member 10. The linear member 20 is easily fixed to the tubular member before formation of the tapered surface. Forming the tapered surface on the tubular member is thus preferably conducted after fixing the linear member 20 to the tubular member.

The present application claims benefit of priority based on Japanese Patent application No. 2019-019998 filed on Feb. 6, 2019. The entire contents of the specification of Japanese Patent application No. 2019-019998 filed on Feb. 6, 2019 are incorporated in the present application for reference.

### DESCRIPTION OF REFERENCE SIGNS

1 first tapered portion
2 second tapered portion
3 tapered portion
4 tubular portion
5 unreinforced portion
6 reinforced portion
10 distal member
11 inner layer
12 outer layer
13 reinforcing layer
14 radiopaque marker
linear member
21 grip member
30 extension catheter
31 first resin
32 second resin
33 third resin
40 tubular catheter
S1 first tapered surface
S2 second tapered surface
S10 outer surface
A1 distal end of first tapered portion
A2 distal end of second tapered portion
A3 distal end of tapered portion
A30 distal end of extension catheter
A40 distal end of tubular catheter
B1 proximal end of first tapered portion
B2 proximal end of second tapered portion
B3 proximal end of tapered portion
B13 proximal end of reinforcing layer
B40 proximal end of tubular catheter
C central axis of tubular portion
F1 farthest point on first tapered surface from central axis of tubular portion
N1 closest point on first tapered surface to central axis of tubular portion
X axial direction of tubular portion
a30 distal opening of extension catheter
a40 distal opening of tubular catheter
b40 proximal opening of tubular catheter

1. An extension catheter having a proximal side and a distal side, the extension catheter comprising:
a tubular portion;
a tapered portion arranged such that the tapered portion is disposed at the proximal side and the tubular portion is disposed at the distal side; and
a linear member fixed to the tubular portion and the tapered portion;
the extension catheter satisfying Formula (1):
1.1T₂≤T₁≤6T₂ (1)
wherein T₁ represents a thickness (mm) of the tubular portion at a first position located 1 mm to the distal side of a distal end of the tapered portion, the first position being a position where the linear member is present, and T₂ represents a thickness (mm) of the tubular portion at a second position located 1 mm to the distal side of the distal end of the tapered portion, the second position being opposite to the first position;
the extension catheter being configured to be inserted to a tubular catheter and protrude from a distal opening of the tubular catheter.

2. The extension catheter according to claim 1, wherein the linear member is composed of a metal.

3. The extension catheter according to claim 1, wherein
the tubular portion has a part being composed of a resin at the second position.

4. The extension catheter according to claim 1, further satisfying Formula (2):
1.1T₂≤T₁−T₃≤4T₂ (2)
wherein T₁ and T₂ respectively represent the thicknesses as defined in Formula (1), and T₃ represents a thickness (mm) of the linear member at the first position.

5. The extension catheter according to claim 1, further satisfying Formula (3):
1.1T₁≤T₅≤2T₁ (3)
wherein T₁ represents the thickness as defined in Formula (1), and T₅ represents a thickness (mm) of the tapered portion at a third position located 1 mm to the distal side of a proximal end of the tapered portion, the third position being a position where the linear member is present.

6. The extension catheter according to claim 1, further satisfying Formula (4):
1.1T₆≤T₄≤2T₆ (4)
wherein T₄ represents a minimum distance (mm) from the linear member to an inner surface of the tubular portion at the first position, and T₆ represents a minimum distance (mm) from the linear member to an inner surface of the tapered portion at a third position located 1 mm to the distal side of a proximal end of the tapered portion.

7. The extension catheter according to claim 1, further satisfying Formula (5):
1.1(T₅−T₇)≤T₁−T₃≤3(T₅−T₇) (5)
wherein T₁ represents the thickness as defined in Formula (1), T₃ represents a thickness (mm) of the linear member at the first position, T₅ represents a thickness (mm) of the tapered portion at a third position located 1 mm to the distal side of a proximal end of the tapered portion, the third position being a position where the linear member is present, and T₇ represents a thickness (mm) of the linear member at the third position.

8. The extension catheter according to claim 1, further comprising,
at the first position, a first resin disposed outside the linear member, and a second resin disposed outside the first resin, the second resin having a higher melting point than a melting point of the first resin.

9. The extension catheter according to claim 1, further comprising,
at the first position, a first resin disposed outside the linear member, and a second resin disposed outside the first resin, the second resin having a higher Shore hardness than a Shore hardness of the first resin.

10. The extension catheter according to claim 1, further comprising,
at the first position, a first resin disposed outside the linear member, and
a third resin disposed at the second position, the third resin having a lower melting point than a melting point of the first resin.

11. The extension catheter according to claim 1, further comprising,
at the first position, a first resin disposed outside the linear member, and
a third resin disposed at the second position, the third resin having a lower Shore hardness than a Shore hardness of the first resin.

12. The extension catheter according to claim 1, further satisfying Formula (6):
1.1T₉≤T₈≤6T₉ (6)
wherein T₈ represents a thickness (mm) of a part of the tubular portion, the part being adjacent to the linear member and disposed at a fifth position located 0.2 mm to the distal side of a distal end of the linear member, and T₉ represents a thickness (mm) of the tubular portion at a sixth position, the sixth position located at a position opposite to the fifth position.

13. The extension catheter according to claim 1, further satisfying Formula (7):
1.1T₈≤T₅≤2T₈ (7)
wherein T₈ represents a thickness (mm) of a part of the tubular portion, the part being adjacent to the linear member and disposed at a fifth position located 0.2 mm to the distal side of a distal end of the linear member, and T₅ represents a thickness (mm) of the tapered portion at a third position located 1 mm to the distal side of a proximal end of the tapered portion, the position being a position where the linear member is present.

14. The extension catheter according to claim 1, wherein
T₁ is 0.1 mm or more and 0.4 mm or less, and
T₂ is 0.05 mm or more and 0.2 mm or less.

15. A method of producing an extension catheter, the method comprising:
attaching a resin to a linear member and disposing the linear member on a tubular member;
surrounding the linear member and the tubular member with a heat-shrinkable film, shrinking the heat-shrinkable film by heat to fix the linear member to the tubular member; and
forming a tapered surface on the tubular member.
